(19) United States
(12) Patent Application Publication
(10) Pub. No.: US 2012/0238814 A1
(43) Pub. Date: Sep. 20, 2012

(54) GUIDE ASSEMBLY FOR ENDOSCOPE

(76) Inventors: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP)

(21) Appl. No.: 13/408,737

(22) Filed: Feb. 29, 2012

(30) Foreign Application Priority Data
Mar. 15, 2011 (JP) 2011-056345

(51) Int. Cl. A61B 1/00

(52) U.S. Cl. 600/114

(57) ABSTRACT

A guide assembly for an endoscope includes a shaft sleeve, barrel sleeve, support sleeve and endless track device for propulsion by endlessly moving in an axial direction of an elongated tube. A rotation wheel is supported on the barrel sleeve, for driving the endless track device by rotating. An idler roller is supported on the support sleeve in a rotatable manner, for keeping the endless track device movable in cooperation with the rotation wheel. A small diameter portion is formed with two axial ends of the rotation wheel by decreasing a wheel diameter thereof along a wheel axis, for allowing leaving of the idler roller from the rotation wheel upon rotationally shifting the support sleeve around the barrel sleeve, so that the endless track device is released from tight contact therewith, and separated from the barrel sleeve in the axial direction with the support sleeve.

### BACKGROUND OF THE INVENTION

1. Field of the Invention

The present invention relates to a guide assembly for an endoscope. More particularly, the present invention relates to a guide assembly in which a propulsion unit with an endless track device can be easily removed from a driving device for driving the propulsion unit by attachment to the endoscope.

2. Description Related to the Prior Art

An endoscope has an elongated tube or guide tube for entry in a body cavity of a patient. The elongated tube includes a head assembly, a steering device and a flexible tube. The head assembly is positioned at its tip. The steering device steers the head assembly in a direction as desired. The flexible tube extends from the steering device. An imaging window and the like are formed in a distal end of the head assembly for imaging of an object.

Manipulation of the endoscope is a difficult process, because a large intestine in a gastrointestinal tract is a tortuous organ in a human body, and some body parts are very changeable in the position in the body. Learning the manipulation of the endoscope of the large intestine requires much experience and time. If a doctor is insufficiently skilled in the manipulation, physical load to the body of a patient will be very large.

Portions of the large intestine where entry of an endoscope is locally very difficult include a sigmoid colon and transverse colon. Specifically, the sigmoid colon and transverse colon is present in the body at changeable locations unlike other body parts. Their shapes are changeable within ranges of their sizes. Also, the sigmoid colon and transverse colon may be deformed in the body by contact pressure of the endoscope upon its entry. Various ideas of manipulation have been suggested for enabling straight forms of the sigmoid colon or the transverse colon so as to reduce contact a wall of the large intestine upon entry of the endoscope. In view of this, there is a suggestion of a guide assembly or self-propelled apparatus as a structure featured today, which propels the endoscope in a distal direction in an intestine or colon in order to facilitate the entry even with manipulation of an unskilled operator.

The guide assembly disclosed in U.S. Pat. Nos. 6,971,990 and 7,736,300 (corresponding to JP-A 2009-513250) includes a housing, a movable endless track device or crawler device or toroidal device, and a transmission device or driving device. The housing is a sleeve for receiving insertion of an elongated tube of the endoscope. The endless track device is disposed to extend around the housing. The driving device endlessly turns around the endless track device to move the endoscope in the distal or proximal direction as axial direction. The endless track device is formed from resilient material, and is filled with fluid (liquid or gas) for inflation in a manner of a balloon of rubber, for contact with a wall of the intestine at a conditioned pressure. The endoscope can move smoothly through the intestine in contact with the wall.

The endoscope after the use at one time is cleaned up by a washer constructed specially. The guide assembly must be removed from the endoscope and washed. However, according to U.S. Pat. Nos. 6,971,990 and 7,736,300, an outer unit including the endless track device to move in contact with the wall is pressed on an inner unit for attachment to the elongated tube by a pair of rollers which squeeze the endless track device in inward and outward directions. The outer unit in the guide assembly cannot be separated or disassembled from the inner unit. Although the endless track device should be exchanged at each time of the use after direct contact with the wall, it is important to reuse the inner unit by washing because of incorporation of the driving device.

### SUMMARY OF THE INVENTION

In view of the foregoing problems, an object of the present invention is to provide a guide assembly in which a propulsion unit with an endless track device can be easily removed from a driving device for driving the propulsion unit by attachment to the endoscope.

In order to achieve the above and other objects and advantages of this invention, a guide assembly for an endoscope having a section of an elongated tube for entry in a tube of a body cavity is provided. A shaft sleeve has a through lumen, for mounting on the elongated tube. A barrel sleeve is supported around the shaft sleeve. An endless track device has an annular surface, is supported around the barrel sleeve, for contacting an inner wall of the body cavity, and for propulsion by endlessly moving in an axial direction of the elongated tube. A support sleeve is disposed in an inner space of the endless track device. A rotation wheel has a drum shape with a diameter increasing at a center thereof, for moving the endless track device, the rotation wheel being supported on the barrel sleeve in a rotatable manner about an axis extending transversely to the axial direction. At least one idler roller is supported on the support sleeve in a rotatable manner about an axis along the axis of the rotation wheel, and shiftable between an engaged position and a release position by rotation of the support sleeve, wherein the idler roller, when in the engaged position, is aligned with the rotation wheel for keeping the endless track device movable endlessly, and when in the release position, is left from the rotation wheel, to allow removal of the endless track device from the barrel sleeve together with the support sleeve.

The shaft sleeve and the barrel sleeve constitute a driving device, and the support sleeve and the endless track device constitute a propulsion unit.

The idler roller has a peripheral surface with a diameter decreasing at a roller center thereof in correspondence with the drum shape of the rotation wheel.

The idler roller has a radius of curvature of the peripheral surface equal to or more than a radius of curvature of the peripheral surface of the rotation wheel.

Furthermore, a cover is secured outside the support sleeve, for protecting an outer side of the idler roller.

Furthermore, a bias device biases the idler roller toward the rotation wheel.

The rotation wheel is constituted by plural rotation wheels arranged around the barrel sleeve in an angularly equidistant manner from one another, and the idler roller is constituted by plural idler rollers arranged around the support sleeve in an angularly equidistant manner from one another.

The at least one idler roller is two idler rollers disposed on proximal and distal sides from the rotation wheel in the axial direction.

Furthermore, proximal and distal end rings are fitted on ends of the shaft sleeve and the barrel sleeve, for support thereof in keeping a space therein.

The distal end ring is formed together with the barrel sleeve, and the proximal end ring is formed together with the shaft sleeve.

Furthermore, a drive sleeve is disposed between the shaft sleeve and the barrel sleeve, caused to rotate by a drive source, for rotating the rotation wheel.

Furthermore, a proximal end ring is fitted on a proximal end of the shaft sleeve and the barrel sleeve, for support thereof in keeping a space therein. An input gear is supported on the proximal end ring in a rotatable manner, driven by the drive source, for rotating the drive sleeve.

The rotation wheel has teeth. The drive sleeve includes spur gear teeth meshed with the input gear. Worm gear teeth are meshed with the teeth of the rotation wheel.

Furthermore, a control wire transmits rotation of a drive source to the input gear.

In one preferred embodiment, a guide assembly for an endoscope having a section of an elongated tube for entry in a tube of a body cavity is provided. A shaft sleeve has a through lumen, for mounting on the elongated tube by reception in the through lumen. A barrel sleeve is supported around the shaft sleeve. An endless track device has an annular surface, is supported around the barrel sleeve, for contacting an inner wall of the body cavity, and for propulsion by endlessly moving in an axial direction of the elongated tube. A support sleeve is disposed in an inner space of the endless track device. A rotation wheel is supported on the barrel sleeve, for driving the endless track device by rotating. At least one idler roller is supported on the support sleeve in a rotatable manner, for keeping the endless track device movable in cooperation with the rotation wheel. A small diameter portion is formed with at least one of two axial ends of the rotation wheel by decreasing a wheel diameter thereof along a wheel axis, for allowing leaving of the idler roller from the rotation wheel upon rotationally shifting the support sleeve around the barrel sleeve, so that the endless track device is released from tight contact therewith, and separated from the barrel sleeve in the axial direction with the support sleeve.

Thus, a propulsion unit with an endless track device can be easily removed from a driving device for driving the propulsion unit by attachment to the endoscope, because a small diameter portion is formed with at least one of two axial ends of the rotation wheel, for allowing leaving of the idler roller from the rotation wheel upon rotationally shifting the support sleeve.

### BRIEF DESCRIPTION OF THE DRAWINGS

The above objects and advantages of the present invention will become more apparent from the following detailed description when read in connection with the accompanying drawings, in which:

FIG. 1 is a plan illustrating a guide assembly;

FIG. 2 is a vertical section illustrating the guide assembly;

FIG. 3 is an exploded perspective view illustrating the guide assembly;

FIG. 4 is an exploded perspective view illustrating a propulsion unit with a preferred structure of proximal and distal end rings;

FIG. 5 is a plan illustrating a support for idler rollers;

FIG. 6 is a vertical section illustrating the same as FIG. 5;

FIG. 7 is a vertical section illustrating the same as FIG. 2 in enlargement;

FIG. 8 is a cross section illustrating a portion of a relationship between a rotation wheel and the idler roller;

FIG. 9 is a cross section illustrating a portion of the same as FIG. 8 in a release position;

FIG. 10 is a cross section illustrating a portion of an idler roller of a conventional structure;

FIG. 11 is an exploded perspective view illustrating another preferred propulsion unit;

FIG. 12 is a cross section illustrating a portion of a relationship between a rotation wheel and an idler roller with a diameter increasing towards its ends;

FIG. 13 is a cross section illustrating a portion of the same as FIG. 12 in the release position;

FIG. 14 is a vertical section illustrating another preferred embodiment having a cover sleeve.

### DETAILED DESCRIPTION OF THE PREFERRED EMBODIMENT(S) OF THE PRESENT INVENTION

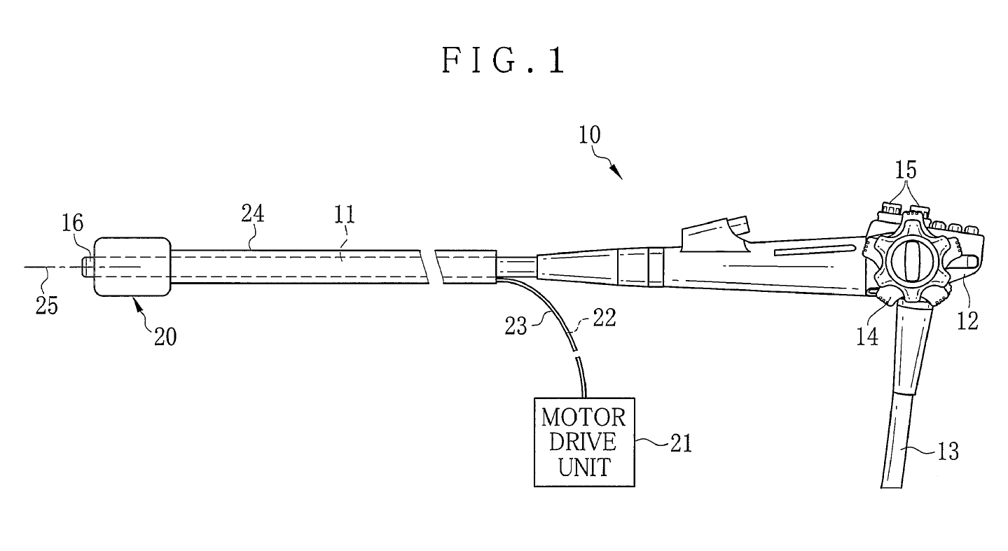

In FIG. 1, an endoscope 10 includes a solid state imaging device such as a CCD or CMOS image sensor of a small type incorporated in a head assembly. The endoscope 10 includes a section of an elongated tube 11 or guide tube, a handle device 12 and a universal cable 13. The elongated tube 11 has the imaging device, and used for entry in a body cavity, such as a large intestine in a gastrointestinal tract. The handle device 12 is manually held and operated for controlling the elongated tube 11. The universal cable 13 extends for connection of the endoscope 10 to a processing apparatus and a light source apparatus (not shown). The universal cable 13 is connected to the handle device 12, and contains a fluid supply channel, a signal cable for an image signal, and a light guide device. The handle device 12 includes steering wheels 14 and fluid supply buttons 15.

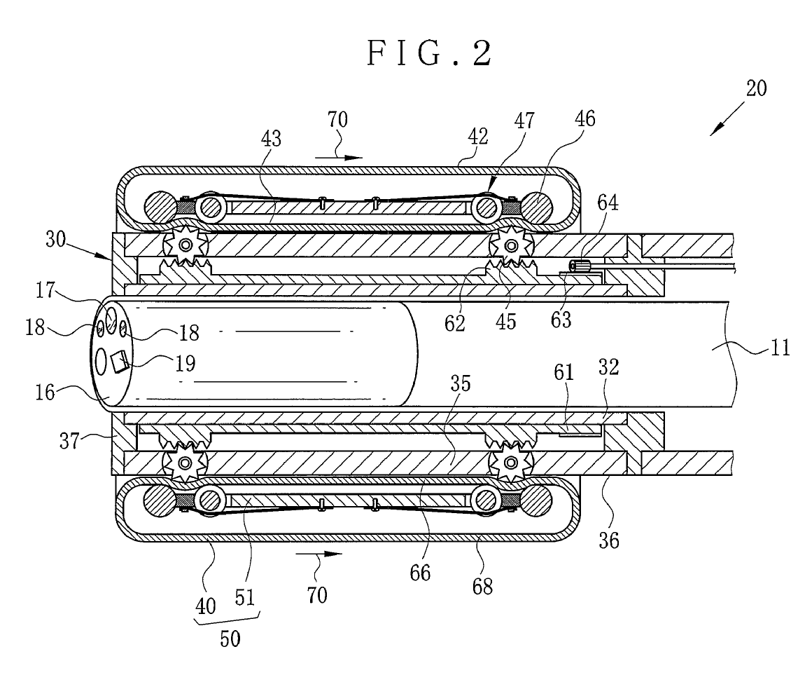

The steering wheels 14 are rotated to set a direction and amount of steering of the elongated tube 11. The fluid supply buttons 15 are used for supply of air or water, suction of fluid and the like. The elongated tube 11 is a flexible element, of which a head assembly 16 is disposed at a distal end. The head assembly 16 includes an imaging window 17, a lighting window 18 and an end nozzle 19. See FIG. 2. A guide assembly 20 or propulsion assembly is mounted on the head assembly 16, and moves the elongated tube 11 in any one of proximal and distal directions in a body cavity.

A motor drive unit 21 or drive source drives the guide assembly 20. A control wire 22 is connected to the motor drive unit 21, and transmits rotational torque for moving the guide assembly 20. A protection sheath 23 covers the control wire 22 with its entire length. The control wire 22 is driven by the motor drive unit 21 to rotate inside the protection sheath 23. A controller (not shown) controls the motor drive unit 21. An input interface is connected to the controller. The input interface includes a control button for inputting a command signal for the guide assembly 20 in relation to advance, return and stop, and a speed button for changing a moving speed of the guide assembly 20.

An overtube 24 externally covers the elongated tube 11. The protection sheath 23 is disposed to extend between the overtube 24 and the elongated tube 11. The overtube 24 has a structure of bellows expandable and compressible in an axial direction 25 of the elongated tube 11.

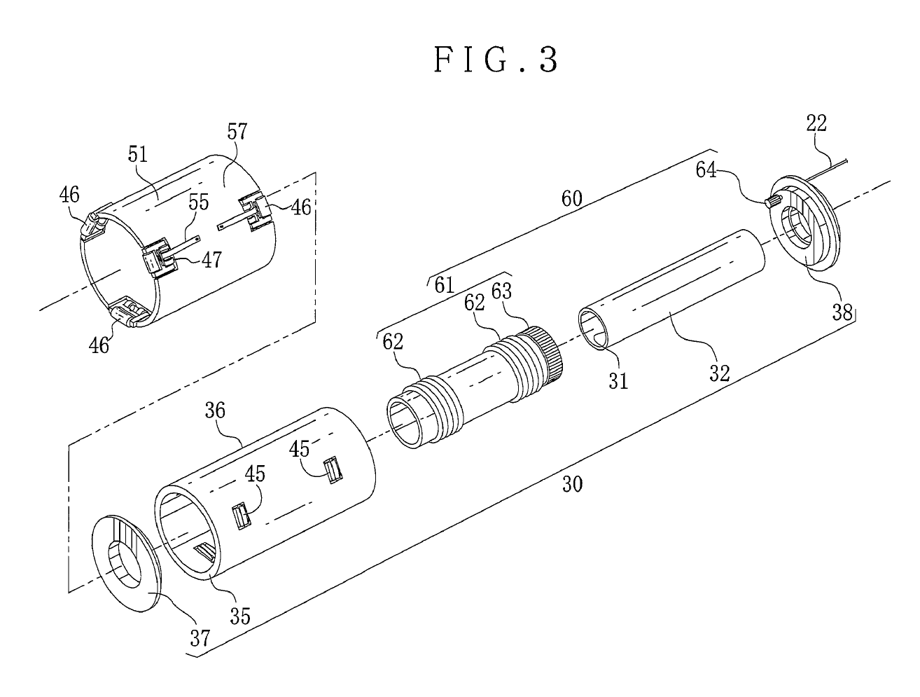

In FIGS. 2 and 3, the guide assembly 20 includes a driving device 30 and a propulsion unit 50 or endless track unit (toroidal unit) . The driving device 30 is mounted on the head assembly 16 of the elongated tube 11 of the endoscope 10. A rotation wheel 45 or engagement roller or driving worm wheel or gear is incorporated in the driving device 30. The propulsion unit 50 includes an endless track device 40 or toroidal device for propulsion, and a support sleeve 51. The endless track device 40 is formed from a plastic sheet of a small thickness, and has expandability and flexibility. The support sleeve 51 is disposed in an inner space of the endless track device 40. Idler rollers 46 and 47 are incorporated in the support sleeve 51. An inner surface 43 of the endless track device 40 is contacted by the idler rollers 46 and 47, which apply tension to the endless track device 40 in a squeezed form with the rotation wheel 45.

The driving device 30 is entered in a center lumen of the endless track device 40. The endless track device 40 includes an upper run 68 (active run) and a lower run 66 (return run). The lower run 66 contacts the driving device 30, and is moved to a distal side in the axial direction of the endoscope 10. Thus, the upper run 68 contacting a wall of a body cavity is moved in a proximal direction 70 opposite to the distal direction. The entirety of the endless track device 40 turns around endlessly for propulsion.

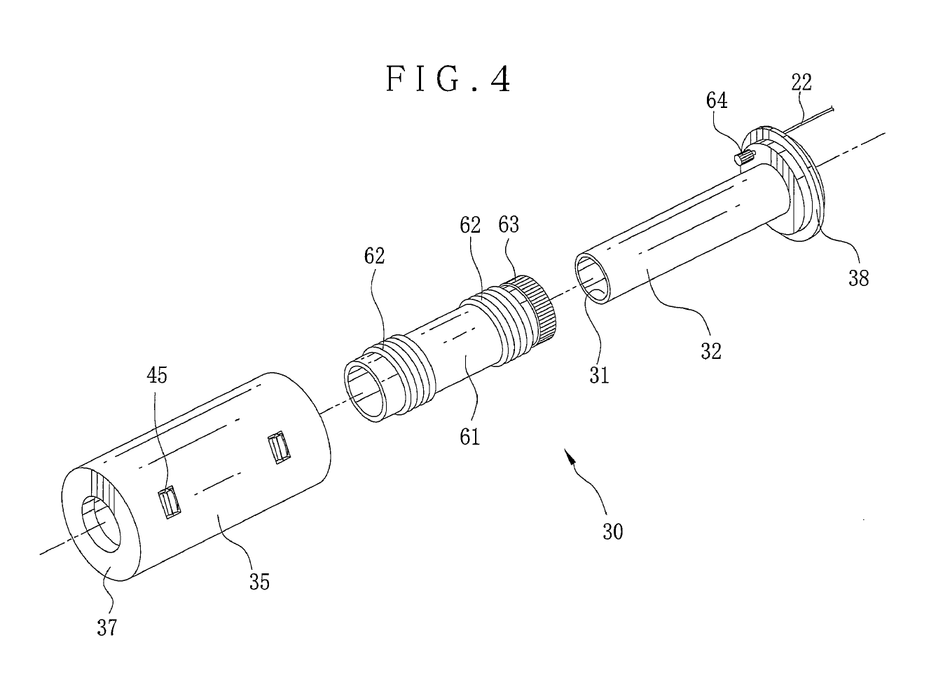

The driving device 30 includes a shaft sleeve 32 (inner sleeve), a barrel sleeve 35 (outer sleeve), a distal end ring 37 and a proximal end ring 38. A through lumen 31 is formed in the shaft sleeve 32, which is mounted on the head assembly 16 of the elongated tube 11 by receiving in the through lumen 31. The proximal and distal end rings 37 and 38 are fitted on the shaft sleeve 32 and the barrel sleeve 35 for support in a coaxial manner with an inner space. Note that the distal end ring 37 may be formed together with the barrel sleeve 35. The proximal end ring 38 may be formed with the shaft sleeve 32. These features are illustrated in FIG. 4.

A gear set 60 or drive mechanism is incorporated in the driving device 30 and applies torque to the rotation wheel 45. The gear set 60 includes a drive sleeve 61 or threaded sleeve, and an input gear 64 or pinion. The drive sleeve 61 is supported around the shaft sleeve 32 in a rotatable manner. The input gear 64 is supported on the proximal end ring 38 in a rotatable manner. The drive sleeve 61 has two worm gears 62 or worm gear teeth (thread), and spur gear teeth 63 at its proximal end. The input gear 64 is disposed between the shaft sleeve 32 and the barrel sleeve 35, and meshed with the spur gear teeth 63. The control wire 22 extends through a through hole in the proximal end ring 38, and connected to the input gear 64, so as to transmit torque of the control wire 22 to the worm gear teeth 62.

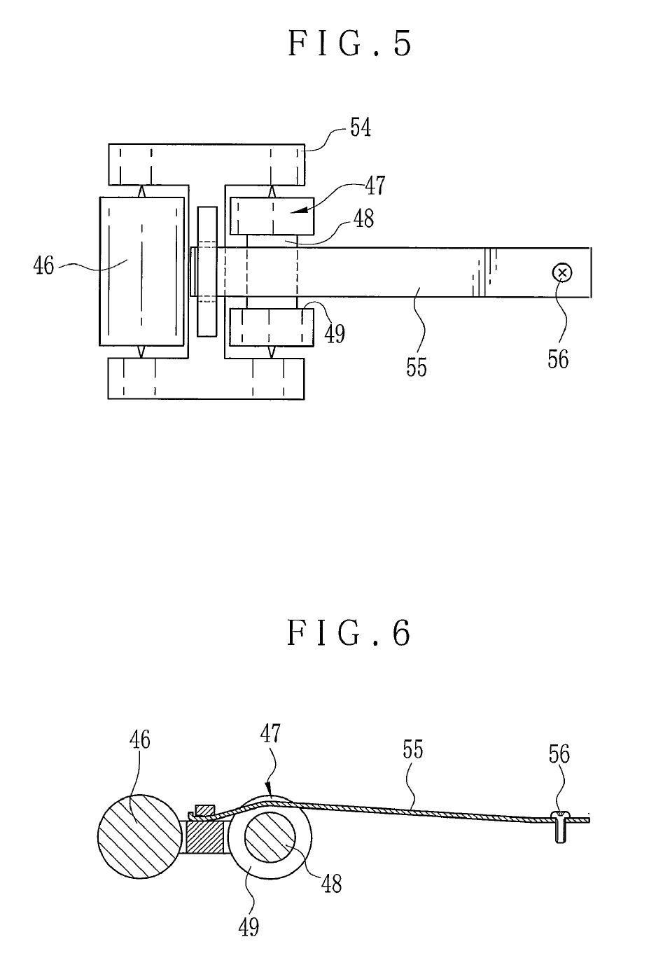

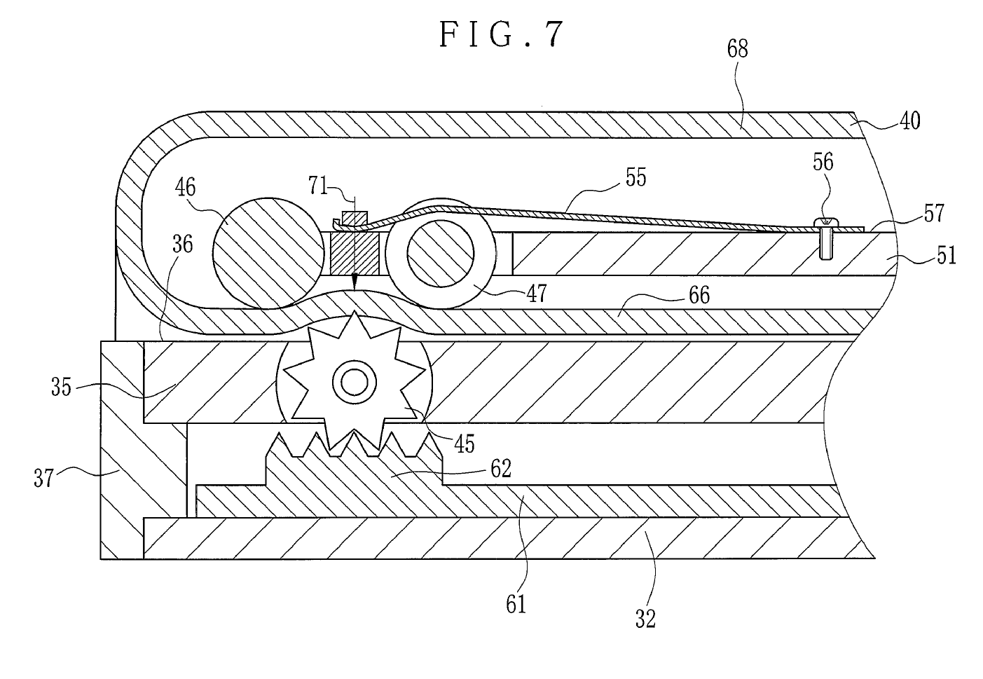

In FIGS. 5, 6 and 7, a roller holder 54 supports the idler rollers 46 and 47 in parallel with one another. A spring plate 55 as a bias device has a first end retained to the roller holder 54 between the idler rollers 46 and 47. A screw 56 fixedly secures a second end of the spring plate 55 to an outer surface 57 of the support sleeve 51. Thus, the idler rollers 46 and 47 are aligned with the rotation wheel 45 in the axial direction 25. The idler rollers 46 and 47 apply tension to the endless track device 40 in a squeezed form with the rotation wheel 45 with inclinations on proximal and distal sides from the rotation wheel 45. A center roll 48 of the idler roller 47 has a small diameter. A recess 49 for a clearance space is defined by the idler roller 47 for movement of the spring plate 55. The spring plate 55 biases the idler rollers 46 and 47 in a transverse direction 71 to press the endless track device 40 on the rotation wheel 45.

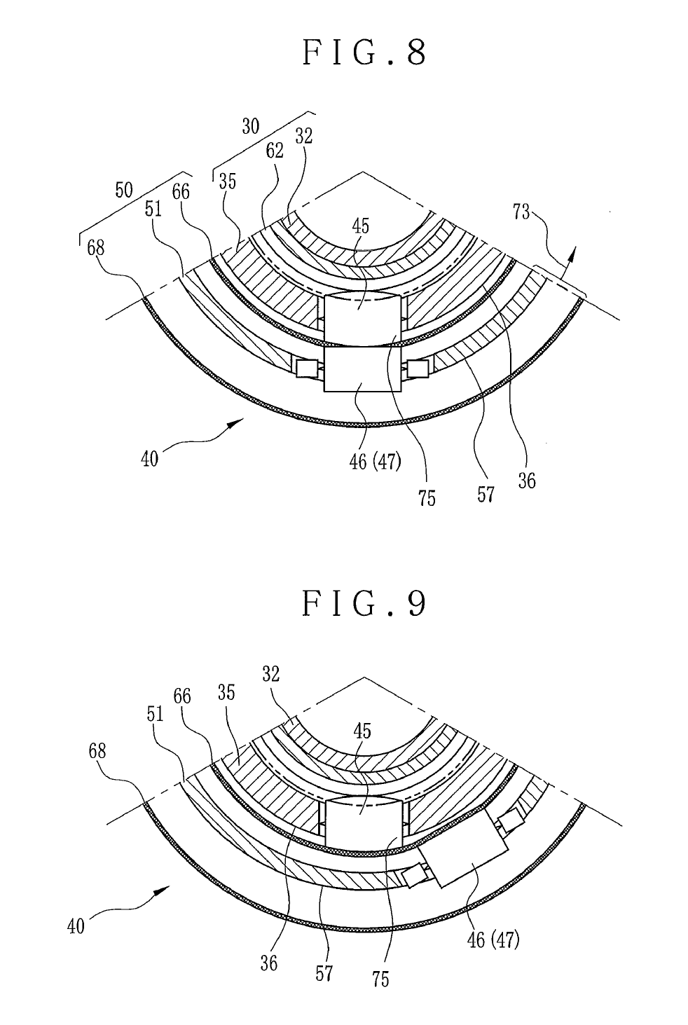

In FIGS. 3 and 8, the barrel sleeve 35 has an outer surface 36. Six rotation wheels 45 are incorporated in the barrel sleeve 35. Among those, three are arranged on the outer surface 36 in a rotationally symmetrical manner at an angular interval of 120 degrees about the axial direction 25. Two among the six are arranged in the axial direction 25 on the barrel sleeve 35. The rotation wheels 45 are in a drum shape with a convexly arcuate curve as viewed in a vertical section, and have small diameter portions 75 at axial ends. Gear teeth with a curved gear tooth profile are present in each of the rotation wheels 45. The gear teeth project to the inside of the barrel sleeve 35, and are meshed with the worm gear teeth 62. The rotation wheel 45 projects to the outside of the barrel sleeve 35, and contacts the endless track device 40. The idler rollers 46 and 47 on the support sleeve 51 apply tension to the endless track device 40 in a squeezed form with the rotation wheel 45, and move the lower run 66 of the endless track device 40 in the axial direction 25, to turn around the endless track device 40 endlessly.

Proximal rollers among the rotation wheels 45 and the idler rollers 46 and 47 draw a proximal portion of the endless track device 40 contacting the barrel sleeve 35. Distal rollers among the rotation wheels 45 and the idler rollers 46 and 47 move out a distal portion of the endless track device 40 contacting the barrel sleeve 35. The distal portion spreads externally, becomes turned over with 180 degrees. An outer surface 42 of the upper run 68 comes in contact with a wall of a body cavity of a body part, such as a large intestine. See FIG. 2.

Also, a proximal end portion of the endless track device 40 in the axial direction is pulled to turn a portion of the outer surface 42 of the upper run 68 (active run) contacting the wall of the body cavity, to bend back into the center lumen of the endless track device 40. Thus, the endless track device 40 operates for propulsion of the guide assembly 20 by moving the lower run 66 (return run) in the distal direction and moving the upper run 68 in the proximal direction. Note that the endless track device 40 can be turned around in a direction reverse to that described above, so as to return the guide assembly 20 in a proximal direction.

The operation of the guide assembly 20 is described. The endoscope 10, after use for endoscopic imaging, is pulled out of the large intestine. The guide assembly 20 is removed from the endoscope 10 and disassembled to remove the propulsion unit 50 from the driving device 30. The driving device 30 is cleaned up together with the endoscope 10 by use of a specific washing apparatus. An unused propulsion unit 50 is combined with the driving device 30 for use in next endoscopic imaging.

In FIG. 8, the guide assembly 20 removed from the endoscope 10 is set in the engaged position where the rotation wheel 45 and the idler rollers 46 and 47 apply tension to the endless track device 40 in a squeezed form with pressure. The driving device 30 cannot be simply pulled away from the propulsion unit 50. The propulsion unit 50 is rotated in a rotational direction 73 of FIG. 8 from outside the endless track device 40. When the propulsion unit 50 is in a release position of FIG. 9, the idler rollers 46 and 47 are left from the rotation wheel 45 by use of the small diameter portions 75. Even though a direction of rotating the propulsion unit 50 is opposite to the rotational direction 73, the rotation wheel 45 can be left from the idler rollers 46 and 47 for separation.

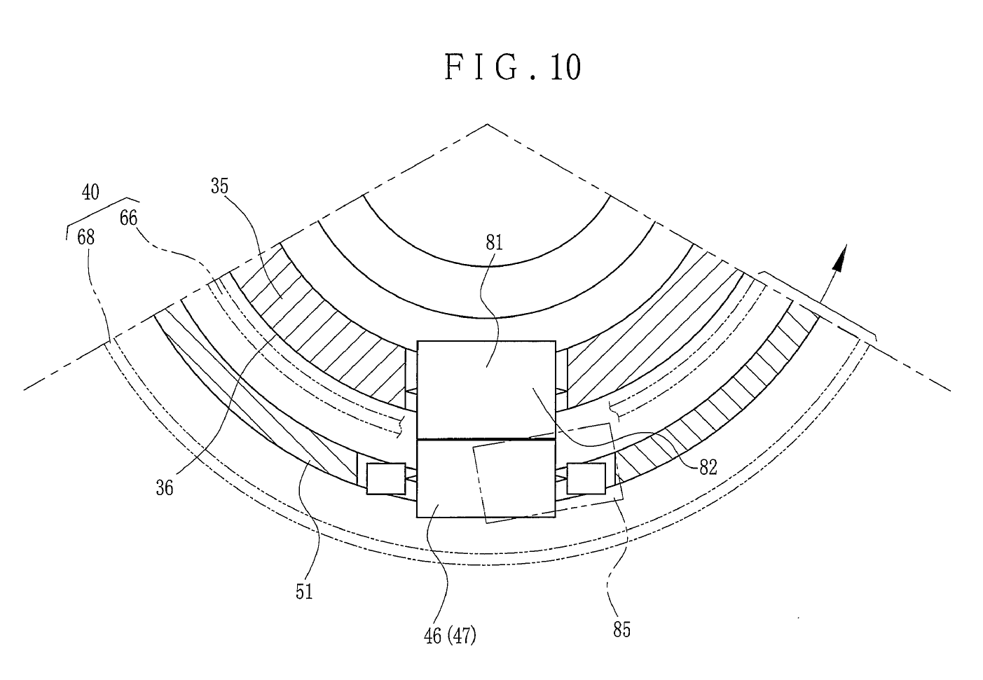

In FIG. 10, a comparative example of a rotation wheel 81 or engagement roller or driving worm wheel or gear is illustrated. If rotational torque is applied to the idler rollers 46 and 47, a corner 82 or edge of the rotation wheel 81 interferes with the idler rollers 46 and 47 at a phantom line 85 for a roller portion. It is impossible to shift the idler rollers 46 and 47 rotationally with the support sleeve 51. The idler rollers 46 and 47 cannot be left or released from the rotation wheel 81. Thus, the driving device 30 cannot be pulled away from the propulsion unit 50.

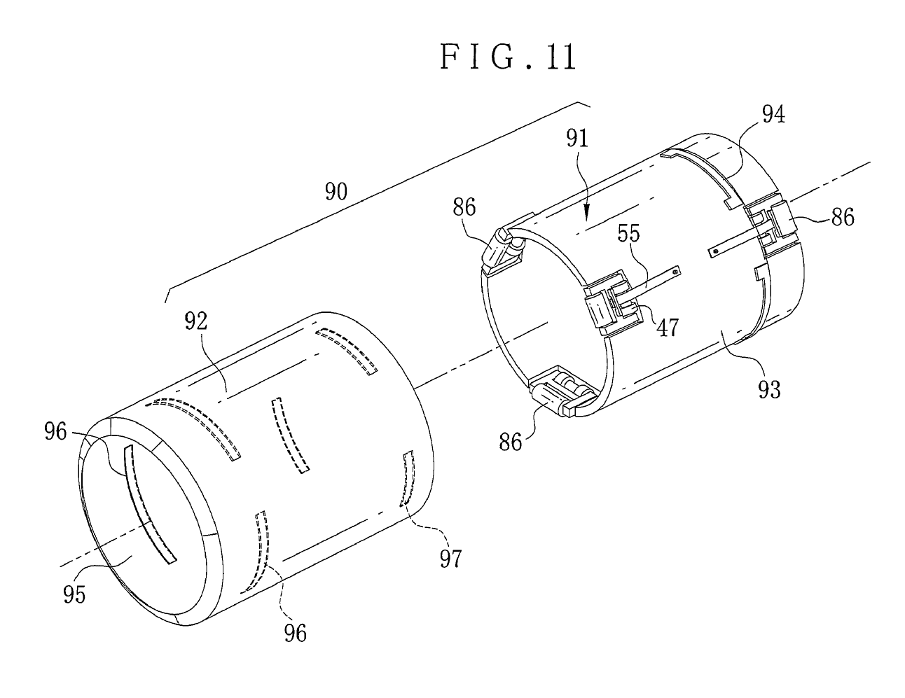

In FIG. 11, another preferred embodiment is illustrated. A propulsion unit 90 or endless track unit (toroidal unit) includes the endless track device 40, a support sleeve 91, and a cover sleeve 92, which is supported around the support sleeve 91.

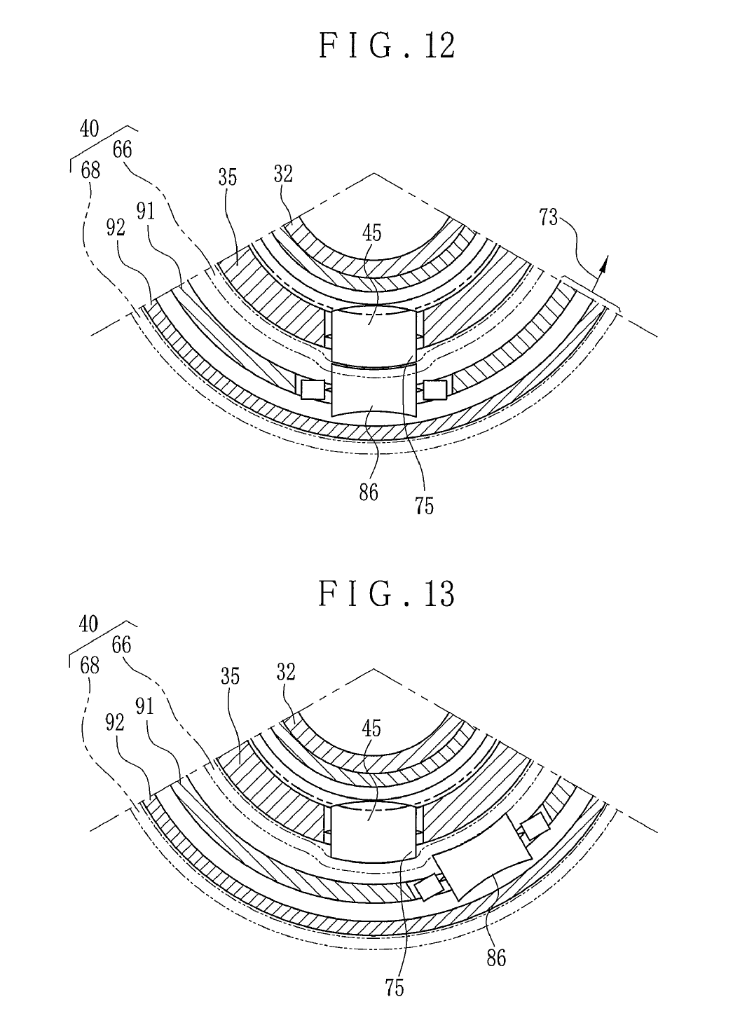

In FIGS. 12 and 13, an idler roller 86 has a concavely arcuate shape as viewed in a vertical section. Preferably, a radius of curvature of the concavely arcuate shape is equal to or slightly more than that of the rotation wheel 45. The idler roller 86 of the concavely arcuate shape exerts higher force of contact to the endless track device 40 in the engaged position of FIG. 12 than the idler roller 46, and exerts force of moving to the release position of FIG. 13 at a level equal to that of the idler roller 46. From this point of view, the idler roller 86 is more preferable than the idler roller 46 for contact with the rotation wheel 45. However, its specific feature must be considered in that end portions of the concavely arcuate shape have a laterally tapered form and should be treated in view of pushing a wall of a body cavity together with the endless track device 40. Thus, the cover sleeve 92 is used with the idler roller 86. Note that if the cover sleeve 92 is not used, it is preferable to form the end portions with a large curvature without a tapered form.

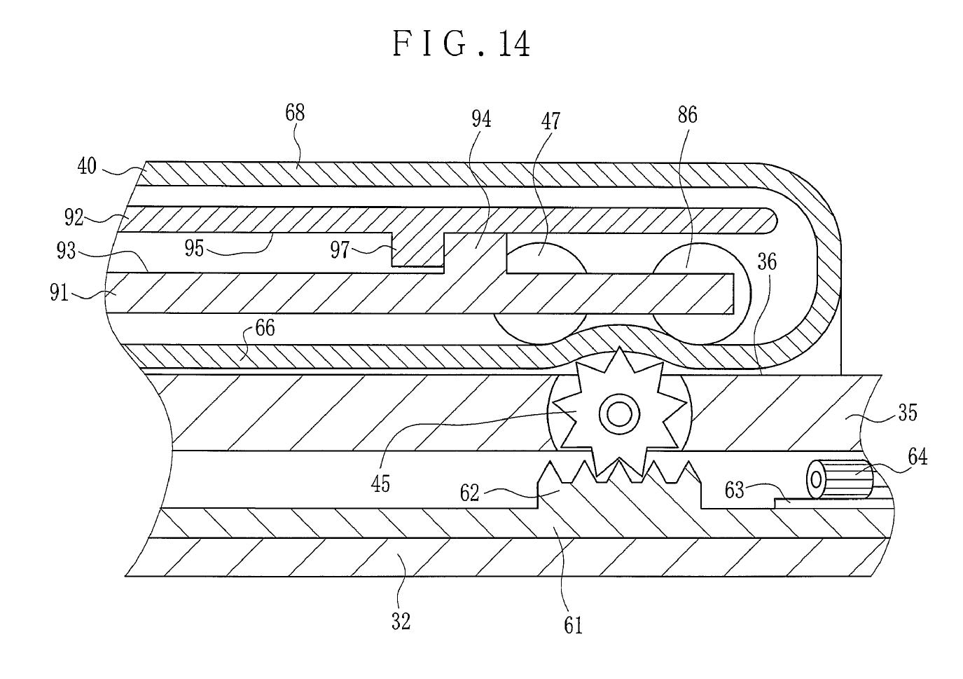

In FIGS. 11 and 14, the cover sleeve 92 includes an inner surface 95 and a spacing projection 96 formed on the inner surface 95 near to its distal end. The support sleeve 91 includes an outer surface 93 and an engaging ridge 94 or support ridge formed on the outer surface 93 near to its proximal end. The spacing projection 96 contacts the outer surface 93. The engaging ridge 94 contacts the inner surface 95, so that the cover sleeve 92 is supported on the support sleeve 91. A receiving ridge 97 or stopper is formed on the inner surface 95 of the cover sleeve 92 and near to its proximal end. The engaging ridge 94 has a U shape of which an open portion is directed to a distal side. The receiving ridge 97 is inserted in the open portion. Three engaging ridges 94 are arranged on the outer surface 93 in a rotationally symmetrical manner at an angular interval of 120 degrees about the axial direction 25, and offset from the spring plate 55. Three spacing projections 96 and three receiving ridges 97 are arranged on the inner surface 95 in a rotationally symmetrical manner at an angular interval of 120 degrees, and offset from the spring plate 55.

The cover sleeve 92 rotates together with the support sleeve 91 owing to the engagement of the engaging ridge 94 with the receiving ridge 97. The support sleeve 91 is rotated by manually rotating the cover sleeve 92 together with the upper run 68 (active run) of the endless track device 40. The idler rollers 46 and 47 are left and released from the rotation wheel 45 so as to enable removal of the driving device 30 from the propulsion unit 90.

Note that screws may be used for securing the support sleeve 91 instead of the engaging ridge 94 and the receiving ridge 97. Also, the cover sleeve 92 maybe formed integrally with the support sleeve 91. In place of using the cover sleeve 92, it is possible to use a cover plate or cover ring attached to the support sleeve 91 for covering the idler rollers 47 and 86.

In the above embodiments, the radius of curvature of the tooth profile of the rotation wheel 45 is determined according to the outer surface 36 of the barrel sleeve 35. However, a radius of curvature of a tooth profile of the rotation wheel 45 can be equal to, or more than or less than the radius of curvature of the outer surface 36 of the barrel sleeve 35.

In the embodiment of FIGS. 11-14, the peripheral surface of the idler roller 86 is in the concavely arcuate shape. A radius of curvature of a peripheral surface of the idler roller 86 can be determined suitably for the purpose, for example, can be smaller than the radius of curvature of the tooth profile of the rotation wheel 45.

In the above embodiments, the idler rollers 46 and 47 are disposed in an alternate manner with the rotation wheel 45. However, at least one idler roller can be opposed to the rotation wheel 45 for squeezing the endless track device 40.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly for an endoscope having a section of an elongated tube for entry in a tube of a body cavity, comprising:
a shaft sleeve, having a through lumen, for mounting on said elongated tube;
a barrel sleeve supported around said shaft sleeve;
an endless track device, having an annular surface, supported around said barrel sleeve, for contacting an inner wall of said body cavity, and for propulsion by endlessly moving in an axial direction of said elongated tube;
a support sleeve disposed in an inner space of said endless track device;
a rotation wheel, having a drum shape with a diameter increasing at a center thereof, for moving said endless track device, said rotation wheel being supported on said barrel sleeve in a rotatable manner about an axis extending transversely to said axial direction;
at least one idler roller, supported on said support sleeve in a rotatable manner about an axis along said axis of said rotation wheel, and shiftable between an engaged position and a release position by rotation of said support sleeve, wherein said idler roller, when in said engaged position, is aligned with said rotation wheel for keeping said endless track device movable endlessly, and when in said release position, is left from said rotation wheel, to allow removal of said endless track device from said barrel sleeve together with said support sleeve.

2. A guide assembly as defined in claim 1, wherein said shaft sleeve and said barrel sleeve constitute a driving device, and said support sleeve and said endless track device constitute a propulsion unit.

3. A guide assembly as defined in claim 1, wherein said idler roller has a peripheral surface with a diameter decreasing at a roller center thereof in correspondence with said drum shape of said rotation wheel.

4. A guide assembly as defined in claim 3, wherein said idler roller has a radius of curvature of said peripheral surface equal to or more than a radius of curvature of said peripheral surface of said rotation wheel.

5. A guide assembly as defined in claim 3, further comprising a cover, secured outside said support sleeve, for protecting an outer side of said idler roller.

6. A guide assembly as defined in claim 1, further comprising a bias device for biasing said idler roller toward said rotation wheel.

7. A guide assembly as defined in claim 1, wherein said rotation wheel is constituted by plural rotation wheels arranged around said barrel sleeve in an angularly equidistant manner from one another, and said idler roller is constituted by plural idler rollers arranged around said support sleeve in an angularly equidistant manner from one another.

8. A guide assembly as defined in claim 1, wherein said at least one idler roller is two idler rollers disposed on proximal and distal sides from said rotation wheel.

9. A guide assembly as defined in claim 1, further comprising proximal and distal end rings, fitted on ends of said shaft sleeve and said barrel sleeve, for support thereof in keeping a space therein.

10. A guide assembly as defined in claim 9, wherein said distal end ring is formed together with said barrel sleeve, and said proximal end ring is formed together with said shaft sleeve.

11. A guide assembly as defined in claim 9, further comprising a drive sleeve, disposed between said shaft sleeve and said barrel sleeve, caused to rotate by a drive source, for rotating said rotation wheel.

12. A guide assembly as defined in claim 11, further comprising an input gear, supported on said proximal end ring in a rotatable manner, driven by said drive source, for rotating said drive sleeve.

13. A guide assembly as defined in claim 12, wherein said drive sleeve includes:
spur gear teeth meshed with said input gear;
worm gear teeth meshed with said rotation wheel.

14. A guide assembly as defined in claim 12, further comprising a control wire for transmitting rotation of a drive source to said input gear.
